(19) United States
(12) Patent Application Publication
(10) Pub. No.: US 2026/0125848 A1
(43) Pub. Date: May 7, 2026

(54) PRODUCT FOR REDUCTION OF BODY ODOURS AND APPLICATION PROCEDURE

(71) Applicant: Oxylus, D.O.O., Osijek (HR)

(72) Inventor: Ivo Domazet, Osijek (HR)

(21) Appl. No.: 19/117,593

(22) PCT Filed: Sep. 29, 2023
(86) PCT No.: PCT/HR2023/050004

§ 371 Date: Apr. 1, 2025

(30) Foreign Application Priority Data
Oct. 3, 2022 (HR) P20221214A

(51) Int. Cl. D06M 23/12; C12N 9/18; C12N 9/26; C12N 9/48; D06M 11/55; D06M 13/00; D06M 13/123; D06M 13/192; D06M 13/332; D06M 13/352; D06M 13/463; D06M 15/09; D06M 16/00

(52) U.S. Cl. CPC D06M 23/12; C12N 9/18; C12N 9/2414; C12N 9/48; D06M 11/55; D06M 13/005; D06M 13/123; D06M 13/192; D06M 13/332; D06M 13/352; D06M 13/463; D06M 15/09; D06M 16/00; C12Y 302/01001

(57) ABSTRACT

The product for reduction of body odours disclosed herein contains 60 to 98.5% of demineralized water by weight, 0.2 to 5% of cellulose gum by weight, 0.5 to 10% of salt by weight, 0.2 to 5% of preservative by weight, 0.5 to 5% of capsules containing the neutralizer for unpleasant odours by weight, 0.1 to 5% of disinfectant by weight, 0 to 5% of tenside by weight and 0 to 5% of fragrance by weight. The product itself is applied directly to the fabric (e.g. T-shirt) or sports equipment (e.g. sports shoes) and is left to dry. During the drying process, a thin layer of nanoparticles containing an encapsulated neutralizer for unpleasant odours is created. When such items that have been treated with this product are worn, the cracking of nanocapsules occurs due to friction and a discrete fragrance that neutralizes the body odour (bacterial activity) is released.

### FIELD OF INVENTION

The present invention relates to the products for reduction of body odours applied to items that are worn (clothes, footwear, sports props) and to the process of applying these products. Also, with certain modifications, this product can be used as a neutralizer for pet odours, for example in a situation when you don't have time to bathe your pet.

### TECHNICAL PROBLEM

The inventor encountered a problem caused by sweating especially in people who are prone to excessive sweating, the so-called hyperhidrosis disorder and the problem caused by odours developed in the presence of sweat. Namely, in such persons and in general in persons that excessively sweat, e.g. due to extreme heat, due to physical activity etc., due to the presence of bacteria on the human skin and the favourable environment of sweat for their reproduction, there is a rapid multiplication of bacteria and the formation of characteristic odours resulting from bacterial decomposition of sweat. Products available on the market must be applied to the person's skin, which often causes irritation and the protection is not long-lasting, i.e. the clothing on the person once soaked in sweat becomes a source of odours because bacteria from the skin are also transferred through sweat to the clothes. The inventor aimed to find a product that will reduce and neutralize body odours, whereby such product is not applied to the user's skin but instead is applied to clothing, footwear and textiles, and which at the same time will enable, once a piece of clothing is treated with such product, the garment to preserve its properties for a longer period. The goal was to find such a product that is not applied to the skin and that only works in the presence of sweat, so that the contact time between the skin and the product is reduced to a minimum, and that such a product fulfils its main function exactly when sweat appears. An additional function of this product is that it can be used as a neutralizer for pet odours by spraying this product on their hair.

### PRIOR ART

Two general solutions are present regarding the reduction and neutralization of body odour and generally sweating: using deodorant as an odour neutralizer and using antiperspirants—products that prevent sweating.

There are several products on the market which help solve problems with excessive sweating primarily either to neutralize odours or to prevent them from appearing at all. The most basic products are ordinary deodorants that reduce the number of bacteria on the skin surface and remove unpleasant body odour. The more advanced products include antiperspirant deodorants that block sweat glands and reduce the amount of sweating. A further option is to use an antiperspirant cream based on aluminium salts applied to the palms, foot soles and armpits, which then reduces sweating. A 6% to 20% solution of aluminium chloride hexahydrate in absolute ethyl alcohol is also used and it's applied to armpit skin, palms or foot soles, but it must be rinsed.

In addition, there is another agent for the prevention of sweating called botulinum toxin A, which is a type of neurotoxin that prevents sweating, however, it must be injected into the armpits and palms. It is known that the use of this neurotoxin prevents sweating for approximately 5-6 months and after that period its application should be repeated. There are also antiperspirant tablets, but the said also have side effects besides blocking the sweating process, such as dryness of the oral cavity and other mucous membranes.

The existing antiperspirant sprays against strong and excessive sweating on the market provide a maximum of 72-hour protection against sweating and unpleasant odours. They contain a high concentration of aluminium chlorohydrate and aluminium chloride which penetrates deep into the pores and thus provides longer-lasting protection, but not longer than 72 hours. This product is applied in the evening to clean, dry and unirrigated skin. Furthermore, a person must take a shower the next morning to rinse off the product.

Hence, the products for prevention of sweating, i.e. reduction or neutralization of body odour that exist in the prior art are applied directly to the user's skin and are of limited duration. However, the inventor hasn't encountered a product for the reduction of body odours applied to clothing and which provides extremely long-lasting protection after application, so the clothing treated with such a product can continue to perform its function to reduce and neutralize body odours even after two years after the application.

### BRIEF SUMMARY OF THE PRESENT INVENTION

Sweating itself is a physiological process by which the body cools down and regulates its temperature. The sweat that rises to the surface gradually evaporates and thus cools the body. The average active person excretes about 0.5-2.0¹ litres of sweat per day. In some people sweating disorder occurs in the form of excessive sweating, especially in the appearance of body odour, and increased sweating is also present during physical activity, especially during sports activities and in obese people. We sweat more in the summer than in the winter, precisely because of body cooling. Sweat glands secrete sweat to the skin surface, wherein there are two types of sweat glands. First, there are eccrine sweat glands, which are mostly found on the foot soles and palms. Sweat that is excreted by eccrine sweat glands is mostly made up of water and salt. The second type are apocrine sweat glands (also called scent glands) located on the armpit skin, genitals, external auditory canal and nipples. Sweat excreted by apocrine sweat glands contains, besides water and salt, proteins and carbohydrates, and these glands are activated during puberty. ¹ https://www.medicinenet.com/how_much_does_an_average_person_sweat_in_a_day/article.htm

The body odour develops when bacteria break down sweat components into volatile fatty acids and some other components and sweat gets an unpleasant smell.

The most commonly present bacteria on the skin that create odours from sweat are the Corynebacterium, Staphylococcus and Cutibacterium genera. These species' most common ones present on the skin are Corynebacterium striatum, Corynebacterium jeikeium, Corynebacterium bovis and Staphylococcus epidermis. The exact mechanisms of body odour production are not fully known.

The main body odours appear as a result of sweat decomposition into fatty acids such as 3-methyl-2-hexenoic acid (3M2H) and 3-hydroxy-3-methylhexanoic acid (HMHA), but the source of body odours and a consequence of the development of thioalcohol, is namely 3-methyl-3-sulfanylhexan-1-ol (3M3SH).

Sweat control is generally established in two ways. One is by affecting the bacterial growth on the skin and the other is by influencing the sweat glands to either reduce or stop their activity. The present invention does not limit or regulate the activity of sweat glands, but rather affects the products of bacterial sweat decomposition, and primarily the clothes that are in contact with the body are being affected, and usually, the skin is not treated in advance in any way until the moment when the sweat appears. Through physical activity of the person wearing the clothes treated with the product disclosed herein, friction occurs between the parts of the clothes or inside the shoes, and nanocapsules that release the body odour neutralizer are activated. The product for reduction of body odours disclosed herein is intended to neutralize and reduce, i.e. to cover up body odours that occur during physical activities that involve sweating, but also in general in situations when people sweat, and the development of unpleasant odours due to bacterial activity on sweat. The product itself in the form of an aerosol or as a coating is directly applied to the fabric (e.g. T-shirt) or sports equipment (e.g. sports shoes) and is left to dry. During the drying process, a thin layer of nanoparticles containing an encapsulated neutralizer for unpleasant odours is created.

When such an item that has been treated with this product is worn, the cracking of nanocapsules occurs due to friction and a discrete fragrance that neutralizes the body odour (bacterial activity) is released. The product may, but doesn't have to be a biocide (contain disinfectant).

The fragrance is retained on the treated surface at least for a day (24 hours) up to a maximum of 2 years (and longer). The durability of the fragrance, i.e. the nanocapsules, depends on the amount applied to the treated item and the intensity of application. In case of intensive wearing of a certain item such as a sports shirt or extreme physical activity, the capsules maintain minimum 24 hours. In case the same shirt is stored after the treatment, for example, for the next season, the capsules maintain on the fabric until the next usage (approx. 2 years). The main ingredients of this product are water, fragrance, cellulose gum, sodium chloride and preservative. The most commonly used fragrance is either alpha-isomethyl ionone or butylphenyl methylpropional or citronellol. Benzisothiazolinone or laurylamine dipropylenediamine is most commonly used as a preservative. A disinfectant and/or tenside and/or fragrance may also be added. Benzalkonium chloride or glutaraldehyde is commonly used as a disinfectant. Ingredient ratios were determined in such a way that this product for reduction of body odours contains 60 to 98.5% of demineralized water by weight, 0.2 to 5% of cellulose gum by weight, 0.5 to 10% of salt by weight, 0.2 to 5% of preservative by weight, 0.5 to 5% of capsules containing the neutralizer for unpleasant odours by weight, 0.1 to 5% of disinfectant by weight, 0 to 5% of tenside by weight and 0 to 5% of fragrance by weight.

Copper sulfate and/or citric acid can be additionally added to improve the properties. The inventor achieved better results when he added enzymes to this product for reduction of body odours. In some applications, proteolytic enzymes have been added, in some and/or alpha-amylases, and in some and/or lipase enzymes. The said enzymes help in the rapid cleavage of more complex impurity molecules, so the product performance is quicker when the enzymes are applied.

### DETAILED DESCRIPTION OF AT LEAST ONE WAY OF CARRYING OUT THE INVENTION

One possible process of making this product for reduction of body odours is to make a mixture containing 95.82 % of demineralized water by weight, 0.25 % of cellulose gum by weight, 1.50 % of salt by weight, 0.20 % of preservative by weight, 2.00 % of capsules containing the neutralizer for unpleasant odours by weight, 0.20 % of disinfectant by weight, 0.01 % of tenside by weight, 0.01 % of copper sulfate by weight and 0.01 % of citric acid by weight. Typically, benzisothiazolinone and/or laurylamine dipropylenediamine is used as a preservative, while benzalkonium chloride or glutaraldehyde is used as a disinfectant. Tenside is usually a fatty alcohol C9-11 or an ethoxylated and/or propoxylated and/or ethoxylated propoxylated block copolymer. This product for reduction of body odours may in one further embodiment contain copper sulfate and/or citric acid.

In another embodiment, the content of demineralized water is 94.65 % by weight, the cellulose gum content is 0.30 % by weight, the salt content is 1.50 % by weight, the preservative content is 0.20 % by weight, the content of the capsules containing the neutralizer for unpleasant odours is 2.50 % by weight, the disinfectant content is 0.50 % by weight, the tenside content is 0.10 % by weight, the copper sulfate content is 0.10 % by weight, the citric acid content is 0.05 % by weight and the fragrance content is 0.10 % by weight.

In one subsequent embodiment, the content of demineralized water is 92.10 % by weight, the cellulose gum content is 0.40 % by weight, the salt content is 1.50 % by weight, the preservative content is 0.20 % by weight, the content of the capsules containing the neutralizer for unpleasant odours is 3.00 % by weight, the disinfectant content is 1.00 % by weight, the tenside content is 1.00 % by weight, the copper sulfate content is 0.50 % by weight, the citric acid content is 0.10 % by weight and the fragrance content is 0.20 % by weight.

In a further embodiment, the content of demineralized water is 89.05 % by weight, the cellulose gum content is 0.50 % by weight, the salt content is 1.50 % by weight, the preservative content is 0.20 % by weight, the content of the capsules containing the neutralizer for unpleasant odours is 3.50 % by weight, the disinfectant content is 1.50 % by weight, the tenside content is 2.50 % by weight, the copper sulfate content is 0.75 % by weight, the citric acid content is 0.2 % by weight and the fragrance content is 0.30 % by weight.

In a further possible embodiment, the content of demineralized water is 87.80 % by weight, the cellulose gum content is 0.50 % by weight, the salt content is 1.50 % by weight, the preservative content is 0.20 % by weight, the content of the capsules containing the neutralizer for unpleasant odours is 4.00 % by weight, the disinfectant content is 2.00 % by weight, the tenside content is 3.00 % by weight, the copper sulfate content is 0.10 % by weight, the citric acid content is 0.50 % by weight and the fragrance content is 0.40 % by weight. Capsules containing the neutralizer for unpleasant odours can be nanocapsules.

Regarding the application procedure of the product for reduction of body odours, the said is used in such a way that it is applied to a certain garment or footwear or a sports prop made of textiles, which needs to be dried after application in order to form a thin layer of nanocapsules containing the product for reduction of body odours and then the garment, footwear or sports prop is ready for use. Nanocapsules get activated due to physical contact and release the neutralizer for unpleasant odours and the fragrance/perfume.

In some further applications, enzymes are added to this product for reduction of body odours. Proteolytic enzymes are usually added, and in some further applications also/or alpha-amylase enzymes and/or lipase enzymes.

In embodiments of the present invention with enzymes, this product may comprise a mixture of enzymes containing proteolytic enzymes, alpha-amylase, disubstituted alanine amide, propylene glycol, glycerol, sodium formate, and phenoxyethanol.

In one further embodiment, this product for reduction of body odours contains 95.539 % of demineralized water by weight, 0.25 % of cellulose gum by weight, 1.50 % of salt by weight, 0.20 % of preservative by weight, 2.00 % of capsules containing the neutralizer for unpleasant odours by weight, 0.20 % of disinfectant by weight, 0.01 % of tenside by weight, 0.01% of copper sulfate by weight, 0.01 % of citric acid by weight, 0.05 % of proteolytic enzymes by weight, 0.01 % of alpha-amylase by weight, 0.01 % of disubstituted alanine amide by weight, 0.1 % of propylene glycol by weight, 0.1 % of glycerol by weight, 0.001% of sodium formate by weight, and 0.01 % of phenoxyethanol by weight.

In another further embodiment, this product for reduction of body odours contains 94.025 % of demineralized water by weight, 0.30 % of cellulose gum by weight, 1.50 % of salt by weight, 0.20 % of preservative by weight, 2.50 % of capsules containing the neutralizer for unpleasant odours by weight, 0.50 % of disinfectant by weight, 0.10 % of tenside by weight, 0.10 % of copper sulfate by weight, 0.05 % of citric acid by weight, 0.10 % of fragrance by weight, 0.07 % of proteolytic enzymes by weight, 0.05 % of alpha-amylase by weight, 0.05 % of disubstituted alanine amide by weight, 0.2 % of propylene glycol by weight, 0.2 % of glycerol by weight, 0.005 % of sodium formate by weight, and 0.05 % of phenoxyethanol by weight.

In a further embodiment, this product contains 91.093 % of demineralized water by weight, 0.40 % of cellulose gum by weight, 1.50 % of salt by weight, 0.20 % of preservative by weight, 3.00 % of capsules containing the neutralizer for unpleasant odours by weight, 1.00 % of disinfectant by weight, 1.00 % of tenside by weight, 0.50 % of copper sulfate by weight, 0.10 % of citric acid by weight, 0.20 % of fragrance by weight, 0.1 % of proteolytic enzymes by weight, 0.1 % of alpha-amylase by weight, 0.1 % of disubstituted alanine amide by weight, 0.3 % of propylene glycol by weight, 0.3 % of glycerol by weight, 0.007 % of sodium formate by weight and 0.1 % of phenoxyethanol by weight.

In a further embodiment, this product for reduction of body odours contains 87.641 % of demineralized water by weight, 0.50 % of cellulose gum by weight, 1.50 % of salt by weight, 0.20 % of preservative by weight, 3.50 % of capsules containing the neutralizer for unpleasant odours by weight, 1.50 % of disinfectant by weight, 2.50 % of tenside by weight, 0.75 % of copper sulfate by weight, 0.2 % of citric acid by weight, 0.30 % of fragrance by weight, 0.15 % of protease by weight, 0.15 % of alpha-amylase by weight, 0.15 % of disubstituted alanine amide by weight, 0.4 % of propylene glycol by weight, 0.4 % of glycerol by weight, 0.009 % of sodium formate by weight, and 0.15 % of phenoxyethanol by weight.

Also, in another embodiment, this product for reduction of body odours contains 85.99 % of demineralized water by weight, 0.50 % of cellulose gum by weight, 1.50 % of salt by weight, 0.20 % of preservative by weight, 4.00 % of capsules containing the neutralizer for unpleasant odours by weight, 2.00 % of disinfectant by weight, 3.00 % of tenside by weight, 0.10 % of copper sulfate by weight, 0.50 % of citric acid by weight, 0.40 % of fragrance by weight, 0.2 % of proteolytic enzymes by weight, 0.2 % alpha-amylase by weight, 0.2 % of disubstituted alanine amide by weight, 0.5 % of propylene glycol by weight, 0.5 % of glycerol by weight, 0.01 % of sodium formate by weight, and 0.2 % of phenoxyethanol by weight.

In addition to the fact that this product for reduction of body odours is used on humans, it can also be used on pets and is applied directly to the hair of e.g. a dog or a cat or a horse or another pet that has hair. By applying this product pets also have a nice and pleasant smell.

1. A product for reduction of body odours wherein the product comprises 60 to 98.5% demineralized water by weight, 0.2 to 5% cellulose gum by weight, 0.5 to 10% salt by weight, 0.2 to 5% preservative by weight, 0.5 to 5% capsules comprising a neutralizer for unpleasant odours by weight, 0.1 to 5% disinfectant by weight, 0 to 5% tenside by weight, and 0 to 5% fragrance by weight.

2. The product for reduction of body odours of claim 1, wherein the preservative is benzisothiazolinone and/or laurylamine dipropylenediamine.

3. The product for reduction of body odours of claim 1, wherein the disinfectant is benzalkonium chloride or glutaraldehyde.

4. The product for reduction of body odours of claim 1, wherein the tenside is a fatty alcohol C9-11 or an ethoxylated block copolymer and/or a propoxylated block copolymer and/or an ethoxylated propoxylated block copolymer.

5. The product for reduction of body odours of claim 1, further comprising copper sulfate and/or citric acid.

6. The product for reduction of body odours of claim 1, wherein the fragrance is alpha-isomethyl ionone or butylphenyl methylpropional or citronellol.

7. The product for reduction of body odours of claim 1, wherein the product comprises 95.82% demineralized water by weight, 0.25% cellulose gum by weight, 1.50% salt by weight, 0.20% preservative by weight, 2.00% capsules comprising the neutralizer for unpleasant odours by weight, 0.20 % disinfectant by weight, 0.01% tenside by weight, 0.01% copper sulfate by weight, and 0.01% citric acid by weight.

8. The product for reduction of body odours of claim 1, wherein the product comprises 94.65% demineralized water by weight, 0.30% cellulose gum by weight, 1.50% salt by weight, 0.20% preservative by weight, 2.50% capsules comprising the neutralizer for unpleasant odours by weight, 0.50 % disinfectant by weight, 0.10% tenside by weight, 0.10% copper sulfate by weight, 0.05% citric acid by weight, and 0.10% fragrance by weight

9. The product for reduction of body odours of claim 1, wherein the product comprises 92.10% demineralized water by weight, 0.40% cellulose gum by weight, 1.50% salt by weight, 0.20% preservative by weight, 3.00% capsules comprising the neutralizer for unpleasant odours by weight, 1.00 % disinfectant by weight, 1.00% tenside by weight, 0.50% copper sulfate by weight, 0.10% citric acid by weight, and 0.20% fragrance by weight.

10. The product for reduction of body odours of claim 1, wherein the product comprises 89.05% demineralized water by weight, 0.50% cellulose gum by weight, 1.50% salt by weight, 0.20% preservative by weight, 3.50% capsules comprising the neutralizer for unpleasant odours by weight, 1.50 % disinfectant by weight, 2.50% tenside by weight, 0.75% copper sulfate by weight, and 0.2% citric acid by weight.

11. The product for reduction of body odours of claim 1, wherein the product comprises 87.80% demineralized water by weight, 0.50 % cellulose gum by weight, 1.50% salt by weight, 0.20% preservative by weight, 4.00% capsules comprising the neutralizer for unpleasant odours by weight, 2.00 % disinfectant by weight, 3.00% tenside by weight, 0.10% copper sulfate by weight, 0.50% citric acid by weight, and 0.40% fragrance by weight.

12. The product for reduction of body odours of claim 1, further comprising alpha-amylase enzymes, and/or proteolytic enzymes, and/or lipase enzymes.

13. The product for reduction of body odours of claim 12, further comprising a mixture of enzymes comprising proteolytic enzymes, alpha-amylase, disubstituted alanine amide, propylene glycol, glycerol, sodium formate, and phenoxyethanol.

14. The product for reduction of body odours of claim 13, wherein the product comprises 95.539% demineralized water by weight, 0.25% cellulose gum by weight, 1.50% salt by weight, 0.20% preservative by weight, 2.00% capsules comprising the neutralizer for unpleasant odours by weight, 0.20 % disinfectant by weight, 0.01% tenside by weight, 0.01% copper sulfate by weight, 0.01% citric acid by weight, 0.05% proteolytic enzymes by weight, 0.01% alpha-amylase by weight, 0.01% disubstituted alanine amide by weight, 0.1% propylene glycol by weight, 0.1% glycerol by weight, 0.001% sodium formate by weight, and 0.01% phenoxyethanol by weight.

15. The product for reduction of body odours of claim 13, wherein the product comprises 94.025% demineralized water by weight, 0.30% cellulose gum by weight, 1.50% salt by weight, 0.20% preservative by weight, 2.50% capsules comprising the neutralizer for unpleasant odours by weight, 0.50 % disinfectant by weight, 0.10% tenside by weight, 0.10% copper sulfate by weight, 0.05% citric acid by weight, 0.10% fragrance by weight, 0.07% proteolytic enzymes by weight, 0.05% alpha-amylase by weight, 0.05% disubstituted alanine amide by weight, 0.2% propylene glycol by weight, 0.2% glycerol by weight, 0.005% sodium formate by weight, and 0.05% phenoxyethanol by weight

16. The product for reduction of body odours of claim 13, wherein the product comprises 91.093% demineralized water by weight, 0.40% cellulose gum by weight, 1.50% salt by weight, 0.20% preservative by weight, 3.00% capsules comprising the neutralizer for unpleasant odours by weight, 1.00 % disinfectant by weight, 1.00% tenside by weight, 0.50% copper sulfate by weight, 0.10% citric acid by weight, 0.20% fragrance by weight, 0.1% proteolytic enzymes by weight, 0.1% alpha-amylase by weight, 0.01% disubstituted alanine amide by weight, 0.3% propylene glycol by weight, 0.3% glycerol by weight, 0.007% sodium formate by weight, and 0.10% phenoxyethanol by weight.

17. The product for reduction of body odours of claim 13, wherein the product comprises 87.641% demineralized water by weight, 0.50% cellulose gum by weight, 1.50% salt by weight, 0.20% preservative by weight, 3.50% capsules comprising the neutralizer for unpleasant odours by weight, 1.50 % disinfectant by weight, 2.50% tenside by weight, 0.75% copper sulfate by weight, 0.20% citric acid by weight, 0.30% fragrance by weight, 0.15% proteolytic enzymes by weight, 0.15% alpha-amylase by weight, 0.15% disubstituted alanine amide by weight, 0.4% propylene glycol by weight, 0.4% glycerol by weight, 0.009% sodium formate by weight, and 0.15% phenoxyethanol by weight.

18. The product for reduction of body odours of claim 13, wherein the product comprises 85.99% demineralized water by weight, 0.50% cellulose gum by weight, 1.50% salt by weight, 0.20% preservative by weight, 4.00% capsules comprising the neutralizer for unpleasant odours by weight, 2.00 % disinfectant by weight, 3.00% tenside by weight, 0.10% copper sulfate by weight, 0.50% citric acid by weight, 0.40% fragrance by weight, 0.2% proteolytic enzymes by weight, 0.2% alpha-amylase by weight, 0.2% disubstituted alanine amide by weight, 0.5% propylene glycol by weight, 0.5% glycerol by weight, 0.01% sodium formate by weight, and 0.20% phenoxyethanol by weight.

19. The product for reduction of body odours of claim 1, wherein the capsules comprising the neutralizer for unpleasant odours are nanocapsules.

20. A method of reducing body odours comprising the steps of applying the product of claim 1 to a garment comprising a fabric, and drying the fabric, whereby the product forms a thin film layer on the fabric of the garment during drying.

21. that the method of claim 20, wherein the product is applied directly to the fabric by spraying or coating.
